(19) United States
(12) Patent Application Publication
(10) Pub. No.: US 2023/0321352 A1
(43) Pub. Date: Oct. 12, 2023

(54) DRUG PREPARATION AND DELIVERY DEVICE

(71) Applicant: EVEON, Montbonnot-Saint-Martin (FR)

(72) Inventors: Vincent DELOBELLE, Montbonnot-Saint-Martin (FR); Adrien BOUILLET, Montbonnot-Saint-Martin (FR); Mahutin AKLE, Montbonnot-Saint-Martin (FR)

(73) Assignee: EVEON, Montbonnot-Saint-Martin (FR)

(21) Appl. No.: 17/999,016

(22) PCT Filed: May 20, 2021
(86) PCT No.: PCT/FR2021/050908

§ 371 Date: Nov. 16, 2022

(30) Foreign Application Priority Data
May 20, 2020 (FR) 2005330

(51) Int. Cl. A61M 5/19; A61M 5/315; A61M 5/142

(52) U.S. Cl. CPC A61M 5/19; A61M 5/142; A61M 5/31596

(57) ABSTRACT

The present invention relates to a drug preparation and drug delivery device comprising: a drug preparation unit comprising at least two receptacles capable of containing constituents to be mixed, at least one of the receptacles being movable inside the drug preparation unit, a drug administration means, such as an injection or spray means, a fluid circuit capable of fluidically connecting the at least two receptacles and the administration means, the connecting of the fluid circuit with the at least two receptacles being made using at least two piercing means when said at least two receptacles move from an initial position to a contact position with said at least two corresponding piercing means, an electromechanical assembly for fluidically driving the constituents within the fluid circuit, characterised in that said drug preparation unit comprises a release mechanism comprising a compressed drive spring which, when it is released, is capable of generating a movement along an axis A1 of at least one of the movable receptacles towards at least one of said piercing means in order to create the fluid connection with the fluid circuit.

### FIELD OF THE INVENTION

The present invention relates to a simplified drug preparation and drug delivery device. The device allows connecting one or more receptacle(s) of primary constituents to a fluid circuit in an automated manner.

The fluid circuit of the invention allows performing a reconstitution and/or an injection of the drug contained in constituent receptacles. Furthermore, the automated device according to the invention can activate electronic functions for starting the drug preparation and drug delivery mechanism and possibly the connection of the power supply battery for example.

### PRIOR ART

Conventionally, the reconstruction of drug requires two vials (one for a lyophilized drug and one for a diluent), a syringe and two needles. This requires training and experience. Therefore, a patient might be at risk of under or overdosing in the event of improper use or mishandling, which might be dramatic depending on the reconstituted drug. Manufacturers will sometimes fill the vials with excess up to 35% in order to compensate for drug losses due to the inability to recover the entire dose, this might lead to excessive or inaccurate dilution prior to administration to the patient.

Some existing products can provide professionals and non-professionals with safe, practical and easy-to-use systems to reconstitute and administer injectable formulations. These systems are provided either as versatile solutions or as components for specialized use. Many reconstitution systems can be customized to currently marketed drugs without changing the manufacturing processes or the packaging components.

They are provided in the form of total systems that can be packaged with filled drug vials and reconstitution components. In general, these systems consist of a plastic device that connects a drug vial to a diluent receptacle, which may be a prefilled syringe, a vial or an infusion bag. However, these solutions do not allow addressing problems of use under difficult conditions for the user, or for non-trained users, and possibly for which the preparation of an injection remains too complex (pediatrics, geriatrics, disabilities, etc.).

To solve this problem, a second approach has emerged with the development of dual chamber syringes that provide a lyophilized drug and a diluent in a single unit. The reconstruction is achieved by pressing on the plunger of the syringe, forcing the diluent through a channel and into the second chamber, where it mixes with the drug to create a solution. Afterwards, the drug may be injected with an attached needle or may be transferred via a so-called Luer connection. These systems offer many advantages for the end user but are nevertheless not very widespread given regulatory and industrial impacts as regards production methods related to this new primary receptacle. The involvement of the patient on a delicate gesture implies a new risk of preparation error.

Thus, the document EP2822526 is known disclosing a drug reconstitution device comprising a disposable liquid transfer unit and a reusable control and drive unit removably connectable to the liquid transfer unit, the liquid transfer unit comprising a housing, a docking interface configured to couple first and/or second constituent element receptacles to the housing, and a pump motor and a fluid flow circuit configured to transfer liquid from the first constituent element receptacle to the second constituent element receptacle. The control and drive unit comprises a pump drive configured to drive the pump motor during a liquid transfer action, the pump drive comprising a transmission output coupling removably engaged with a transmission input coupling of the pump motor when the liquid transfer unit and the control and drive unit are connected. However, this described solution is a preparation platform, which requires recovering the primary receptacle in which the drug has been reconstituted and subsequently using a syringe for carrying out the transfer. This device requires that the user inserts the receptacles by himself into the device: i.e. he applies a sufficiently strong force to pierce the septa, inserts the receptacles in the correct order, then activates the device. If the reconstitution is automated, ignition and final administration still involve human intervention.

An alternative is also offered by the document EP2654938 which discloses a device for mixing at least two constituents originating from at least two separate reservoirs. It comprises at least two distinct orifices able to be connected to said reservoirs, a motor pump, a fluid selector comprising at least one network of through channels able to communicate at least one of said orifices and said pump, said fluid selector being arranged so as to be movable between several positions to selectively allow, by means of said pump, a transfer of at least one constituent from one orifice to the other orifice or to said pump and a mixing of said obtained mixture. This system does not address the technical problems that might be caused by a self-injector device preloaded for a long period of time before use thereof on a patient.

Hence, there is no device addressing the need for automation of the reconstitution and allowing for a reliable fluidic mixing with the strict minimum intervention of the user.

Nor is there any device that is compatible with any combination of receptacles while enabling control of the speed, the time and the fluidic mixing. Indeed, under some particular conditions such as pathologies related to medical emergencies (anaphylactic shock, epileptic seizures, myocardial infarction, cerebrovascular accident) may be sought to reduce the preparation times in order to limit errors or improper uses related to stress and/or to improve the performance through a time saving when taking care of the patients.

A device enabling the automation and the standardization of the preparation and administration of drugs, using primary receptacles in a “so-called ready-to-use preloaded” state, simple to use by reducing the human action to its strict minimum, is not disclosed by the prior art.

### SUMMARY

The present invention has been developed in order to solve the aforementioned problems. Thus, the invention relates to a drug preparation and drug delivery device comprising a drug preparation unit comprising at least two receptacles able to contain constituents to be mixed, at least one of the receptacles being movable in said drug preparation unit, a means for administering the drug, such as an injection or spray means, a fluid circuit able to fluidly connect the at least two receptacles and the administration means, the connection of the fluid circuit with the at least two receptacles being done by means of at least two piercing means when said at least two receptacles move from an initial position to a position of contact with said at least two corresponding piercing means, an electromechanical assembly for fluidly driving the constituents within the fluid circuit, the electromechanical assembly (900) being able to activate the fluid circuit (700, 700′), by means of a motor pump (710). The present invention being characterized in that said drug preparation unit comprises a release mechanism comprising a compressed drive spring which, when it is released, is able to generate a movement according to an axis A1 of at least one of the movable receptacles towards at least one of said piercing means to create the fluid connection with the fluid circuit.

Thus, the device according to the invention allows for a viable technological offer regardless of the type of receptacle used.

Furthermore, it is known that the connection of a receptacle to a fluid circuit might be difficult for users with low dexterity, for example injured, ill or old persons, because the fluid connection throughout a needle might require significant force. In the present invention, the device is designed so as to simplify the action of the user and reduce the force or torque required to cause the fluid connection.

In a preferred embodiment, the drug preparation and drug delivery device according to the invention further comprises a motor pump connected to said fluid circuit for circulating at least one of the constituents in said fluid circuit once the fluid connection is created.

Preferably, the drug preparation and drug delivery device according to the invention is such that, the drug preparation unit comprises: a cradle comprising a first planar wall perpendicular to an axis A1 parallel to the movement of said receptacles and, a carriage movably mounted in said drug preparation unit and abutting on the at least one of the receptacles, said carriage comprising a second planar wall parallel to the first planar wall of the cradle, said drive spring being compressed between said first planar wall of the cradle and said second planar wall of the carriage so that the release of the compressed drive spring moves the at least one receptacle by means of the carriage towards at least one of said piercing means.

Thus, the prior art generally uses prefilled syringes, making the presence of a fluidic system unnecessary because the prefilled syringe is the fluidic system. The invention allows preserving the quality of the drug in the receptacle until the time of reconstitution. Said reconstitution being obtained thanks to the movable carriage enabling the movement of the receptacle towards the fluid circuit only when necessary.

In another preferred embodiment, the drug preparation and drug delivery device according to the invention is such that the release mechanism comprises: a receptacle support extending in the direction A1 coaxially with the compressed drive spring, said support comprising at least two elastic means for fastening said receptacles, an intermediate part removably securing the receptacle support to the compressed drive spring, said attachment being done using an elastic clamp belonging to the receptacle support and elastically retaining a first push rod belonging to said intermediate part, so that the force exerted by the spring necessary to detach the intermediate part and the receptacle support is greater than or equal to the force necessary to move at least one of the movable receptacles.

Therefore, the at least two receptacles carried by the receptacle support are first moved simultaneously and then, once the fluid connection has been obtained, detachment can take place.

In a preferred embodiment, at least one of the two receptacles is closed by a shutter able to move under the effect of an external push in order to expel its constituent, and wherein, said intermediate part comprises a second push rod extending parallel to the thrust axis A1 of the receptacles along said axis A1 of the shutter 620 in order to exert a force on said shutter 620 after detachment between the intermediate part 460 and the receptacle support 450.

The push on the stopper after detachment allows assisting with emptying the constituent contained in the receptacle with a shutter. Above all, by using carpules as receptacle, the reconstitution is possible independently of the orientation of the device while enabling a fluid mixing and not a simple transfer of constituents.

According to another preferred embodiment, the drug preparation and drug delivery device according to the invention comprises an activation unit able to occupy an inactive position in which the compressed drive spring is held and an active position in which the compressed drive spring is released. Thanks to the device according to the invention, in a single action, it is possible to obtain the fluid connection and the electronic activation enabling the reconstitution and possibly the distribution. This complete automation is particularly suited to emergency applications or use in a stress situation, and possibly to use by persons with disabilities.

In an alternative according to the invention, the drug preparation and drug delivery device is such that the activation unit comprises a retaining element able to be driven by said activation unit from a position for holding the spring in its compressed state to a position for releasing the spring, said position for releasing the spring being obtainable by a rotation or a translation of said activation unit by a user. This allows for an easier activation because it requires a minimum of effort and operations.

In another embodiment, the drug preparation and drug delivery device according to the invention is such that the activation unit comprises an electronic board provided with an optical, magnetic or contact type electronic switch, able to wake up the electromechanical assembly for motorized driving of the pump.

Thus, in a single action, it is possible to obtain the fluid connection and the electronic activation enabling the reconstitution and possibly the delivery.

According to another embodiment, the drug preparation and drug delivery device according to the invention is such that the fluid circuit comprises a network of fluid channels extending in a plane and connected to the at least two receptacles and the administration means via channels to reduce bulk in the device.

According to one alternative, the drug preparation and drug delivery device according to the invention is such that the fluid circuit comprises a network of fluid channels extending at the periphery of a central part so as to also reduce bulk and reduce the dead volumes in the fluid path.

According to a preferred embodiment, the drug preparation and drug delivery device according to the invention is such that a first assembly including the fluid circuit, the at least two receptacles and the administration means is disposable whereas a second assembly including the release mechanism, the motor pump and the electromechanical assembly (900) for driving thereof is reusable.

This allows for a high modularity in terms of use since the disposable assembly can be designed as a consumable that is associated with the reusable assembly.

According to a preferred embodiment, the drug preparation and drug delivery device according to the invention is such that it comprises a fluid selector able to selectively connect the at least two receptacles (600) and the administration means (721).

The invention also relates to a method for automated preparation and delivery of a drug using a device as described before, the method comprising the following steps: providing a drug preparation unit comprising at least two receptacles, one comprising a drug in a hydrated or dehydrated form and the other one a solvent, providing a means for administering the drug, such as an injection or spray means, providing a fluid circuit able to fluidly connect the at least two receptacles and the administration means, the connection of the fluid circuit with the at least two receptacles being done by means of at least two piercing means when said at least two receptacles move from an initial position to a position of contact with said at least two corresponding piercing means, providing an electromechanical assembly for fluidly driving the constituents within the fluid circuit, characterized in that the automated preparation and the delivery of the drug comprises the release of a release mechanism comprising a compressed drive spring which, when it is released, generates a movement according to an axis A1 of at least one of the movable receptacles towards at least one of said piercing means to create the fluid connection with the fluid circuit.

According to a preferred embodiment, the method for automated preparation and delivery of a drug according to the invention comprises a step in which electronic activation causes the fluidic drive of the constituents within the fluid circuit and the administration of the drug.

Thus, in a single action, it is possible to obtain the fluid connection and the electronic activation enabling the reconstitution and possibly the delivery. This complete automation is particularly suitable to emergency applications or use in a stress situation, and possibly to use by persons with disabilities.

According to another preferred embodiment, the method for automated preparation and delivery of a drug according to the invention comprises a step in which the electronic activation is done during the movement of at least one of the movable receptacles by means of a switch over the movement path of said receptacles.

Thus, in a single action, it is possible to obtain the fluid connection and the electronic activation enabling the reconstitution and possibly the delivery.

Hence, the invention allows standardizing drug preparation and injection operations in order to ensure the safety of the patient, the quality of the reconstitution, and limit the costs/loss and waste of pharmaceutical active ingredients. It has a particular interest for products sensitive by their instability in liquid form, and having to be reconstituted rapidly before injection: this kind of device allows dividing the preparation time before injection by at least three.

The automation thus obtained allows for a greater reliability of the operation, limiting product losses, bringing the treatment closer to the patient by allowing doing without caregivers and administering the treatments at home for example.

### DEFINITIONS

In the present invention, the terms hereinbelow are defined as follows:
“shutter” indicates a stopper serving as a movable bottom for a receptacle in the device according to the invention.
“substantially” in the context of the invention means that it is within the margin of error corresponding to the accuracy of the tool for measuring the value.
“septum”: in the context of the invention means a diaphragm allowing sealing a tubing or a receptacle.
“carriage”: in the context of the invention means an element whose translational movement causes that of the receptacles that are secured thereto.
“solvent”: in the context of the invention, a solvent should be understood according to its current definition, namely: a liquid having the property of dissolving some substances. It should be noted that in the context of the invention, any previously pumped body fluid should be considered as a solvent, any other active substance in liquid form is also a solvent in the context of the invention.
“carpule”: a term originating from the combination of the cartridge and ampoule words, these are receptacles pre-filled with necessary drugs, easy to set up in the device according to the invention. Said receptacles, called carpules, are tubes provided with elastic stoppers serving as a movable bottom.

### BRIEF DESCRIPTION OF THE FIGURES

The invention will be better understood, and other aims, details, features and advantages thereof will appear more clearly upon reading the following detailed explanatory description of embodiments of the invention provided as purely illustrative and non-limiting examples, with reference to the appended schematic drawings.

In these drawings:

FIG. 1A is an overall perspective view of a device according to the invention,

FIG. 1B is an overall front view of the device of FIG. 1A,

FIG. 1C is an exploded view of the device according to the invention,

FIG. 2A is a perspective view of a first embodiment according to the invention of a drug preparation unit and of a fluid circuit,

FIG. 2B is a perspective view of a second embodiment according to the invention of a drug preparation unit and of a fluid circuit,

FIG. 2C is a front view of the second embodiment of FIG. 2B allowing for a better understanding of the supports of the compressed drive spring,

FIG. 3 is a partial illustration with enlargement of the release mechanism according to the invention of FIG. 2B,

FIG. 4A is a perspective view of a coupling of a planar fluid circuit and of a pump according to the invention,

FIG. 4B is a cross-sectional perspective view of a coupling of a planar fluid circuit and of a pump according to the invention showing the internal fluid channel network of the circuit,

FIG. 4C is a partial cross-sectional view according to the axis XX of the coupling of a fluid circuit and of a pump according to FIG. 4B,

FIG. 5A is a perspective view of a coupling of a three-dimensional fluid circuit and of a pump according to the invention,

FIG. 5B is a plan view of a section according to the axis YY of the coupling of a three-dimensional fluid circuit and of a pump of FIG. 5A,

FIG. 6A is a perspective view of an activation unit according to the invention,

FIG. 6B illustrates an activation unit according to the invention with an enlargement of the spring retaining element.

### DETAILED DESCRIPTION

The present invention relates to a device for delivering a liquid product and it will be better understood upon reading the following figures, which are a non-limiting illustration of the invention.

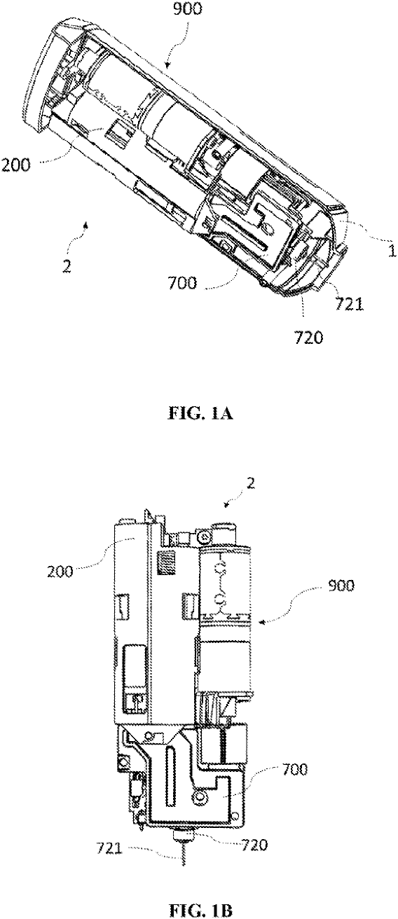

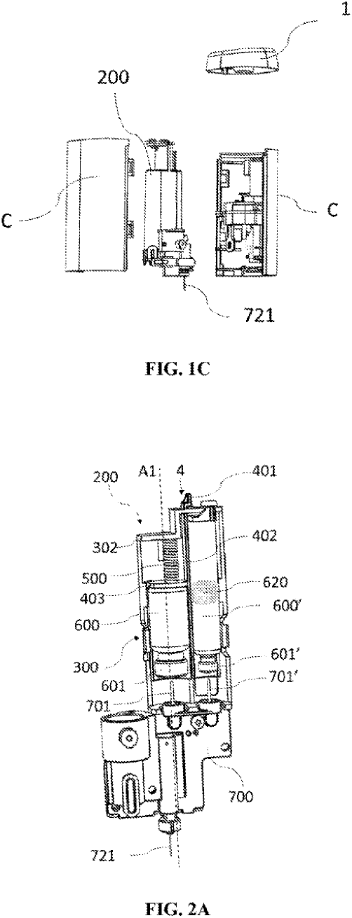

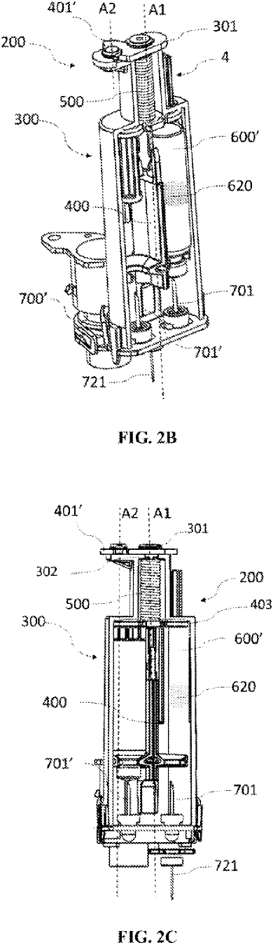

In FIG. 1A, there is shown a drug preparation and drug delivery device 2 with a half-shell. FIG. 1B is a simple front view of the device of FIG. 1A to facilitate understanding. This device is composed of a fluid circuit 700 able to be activated and controlled by an electromechanical assembly 900. This activation is done by means of a motor pump 710, intended to be driven by said electromechanical assembly 900. A drug preparation unit 200 allows connecting primary receptacles 600, 600′ (visible in FIGS. 2A and 2B), at least one of which comprises the drug to be reconstituted and/or injected, to the fluid circuit 700.

The assembly 900 contains at least one motor intended to activate the motor pump 710 and all of the electronic elements including the activation means as described hereinabove. More particularly, the electronic assembly 900 may comprise in particular an optical, magnetic or contact type electronic switch, able to wake up the electromechanical assembly 900 for the motorized drive of the pump 710.

At least one of the primary receptacles 600, 600′ is preloaded. In the case of two drug constituents to be mixed, the two primary receptacles 600, 600′ are preloaded. Thus, they already include the drugs or drug elements intended to be administered. This allows getting rid of the different steps of connection of receptacles which may conventionally require forces greater than 30N (sometimes up to 60N). For some pathologies or some conditions and/or situations (for example a very alienating momentary condition such as a migraine attack, or a stress situation, or medical emergency), or age categories, this stress may become a real brake on the proper progress of the activation of an auto-injector. Therefore, the activation mode and the device 2 according to the present invention aims to make the starting action of the device 2 more reliable.

The entire device is contained in a shell C composed of two complementary portions (cf. FIG. 1C).

The electromechanical assembly 900 and the motor pump 710 are fastened directly or via a support part in the shell C. The fluid circuit 700 may be assembled with the electromechanical assembly 900 or coupled to the shell C. The assembly of the fluid circuit 700 may be permanent or removable in order to be able to discard it if it is soiled and is not intended to be reused. As illustrated in the exploded view of FIG. 1C, the activation unit 1 can be assembled directly on the shell C. It is also possible to consider assembling it directly with the fluid circuit 700. The assemblies may be done with screws, clips, welding, or gluing.

The device according to the invention may include a first disposable assembly including the fluid circuit 700, the motor pump 710 and the receptacles 600, 600′ and a second reusable assembly including the electromechanical assembly 900 able to drive the pump 710 and the fluid circuit 700, the first disposable assembly and the second reusable assembly being arranged so as to be connectable and detachable respectively from one another.

For example, in the case where the device uses two receptacles, the first receptacle may, for example, contain a fluid solvent and the second receptacle may contain a solid powder or a lyophilized product.

In such a case, the advantage of the electromechanical assembly 900 is the ability to parameterize a mixing sequence defined, inter alia, by:
a number of back-and-forth movements between the two primary receptacles 600, 600′, and
the rotational speed of the motor pump 710 to stir the fluidic solvent within the fluid circuit 700.

In the case where only one of the two primary receptacles 600, 600′ includes a drug, this possibly enables the suction of a body fluid before being mixed with the preloaded drug then administered again by the device 2.

The electromechanical unit 900 also allows, by means of the motor pump 710, controlling an accurate dose to be injected, this dose possibly being smaller than the volume of fluid contained in the primary receptacles 600, 600′. Thus, this allows adapting to a wide variety of patients. This confers a certain advantage over devices intended to administer the entire content of their receptacle, for example until the pressure within the receptacle becomes insufficient to expel the content.

The motor pump 710 also allows circulating fluid in the fluid circuit while avoiding any sudden actuation, such as a spring or piston activation, requiring a given force that some patients cannot impart.

FIGS. 2A and 2B illustrate two distinct embodiments of drug preparation units 200.

FIG. 2A illustrates a drug preparation unit 200 including a cradle 300 extending longitudinally along an axis A1. The cradle 300 includes a first planar wall 302 extending perpendicularly to the axis A1.

The drug preparation unit 200 also comprises, within it, a carriage 400 in the form of a planar part extending in a plane parallel to that of the first planar wall 302 of the cradle 300.

A drive spring 500 is compressed between the first planar wall 302 of the cradle 300 and the carriage 400. The drive spring 500 has an axis coincident with the axis A1 of longitudinal extension of the drug preparation unit 200. The accurate operation of the spring is described later on.

On the face opposite to that in contact with the drive spring 500, the carriage 400 bears against the planar base of the first primary receptacle 600, which herein has a cylindrical shape with the axis A1. It should be noted that the first primary receptacle 600 is associated with the carriage 400. Thus, the first primary receptacle 600 is movable within the cradle 300, and therefore within the drug preparation unit 200.

A second primary receptacle 600′ is comprised in the drug preparation unit 200. The two primary receptacles referenced 600, 600′ are in FIG. 2A with a substantially cylindrical general shape but they could have a different section. The two primary receptacles 600, 600′ extend longitudinally along axes parallel to the axis A1.

At its distal end with respect to that in contact with the carriage 400, each primary receptacle 600, 600′ comprises a corresponding septum 601, 601 allowing hermetically closing said primary receptacle 600, 600′. This also allows permanently preserving the preloaded content of each primary receptacle 600, 600′.

### ARRANGEMENT

### First Embodiment

In this first embodiment, the two primary receptacles 600, 600′ are movable. Only the second primary receptacle 600′ comprises a removable bottom, enabling the compression of the fluid.

In FIG. 2A, the second primary receptacle 600′ has a removable bottom in the form of a shutter 620 allowing, when said removable bottom is pushed towards the septum, 601′, to empty the constituent located in the second primary receptacle 600′.

The fluid circuit 700 facing the primary receptacles 600, 600′ is replicated in FIG. 2A. This fluid circuit 700 comprises two needles 701, 701′, said needles extend parallel to one another and coaxially to the axis A1 common to the primary receptacle cylinders 600, 600′ and to the drive spring 500. Preferably, said needles are identical and have as a support a docking port resting on a plane perpendicular to the axis A1.

In the embodiment illustrated in FIG. 2A, the drug preparation unit 200 comprises a release mechanism 4 removably coupled to the compressed drive spring 500 which, when it is released, is able to generate a movement according to the axis A1 of the two movable primary receptacles 600, 600′ towards the needles 701, 701′ to create the fluid connection with the fluid circuit 700. This enables the constituents comprised in the receptacles to circulate in channels of the network. The shutter 620 is able to be pushed to facilitate the discharge, where necessary, of the constituents in the sealed receptacle in a movable manner. The reconstituted mixture can be dispensed using an injection needle 721 after having circulated in the fluid circuit 700.

As already mentioned above, the fluid(s) can circulate in both directions in the fluid circuit 700, allowing for an efficient mixing by several back-and-forth movements.

The release mechanism 4 removably coupled to the compressed drive spring 500 comprises a hooked end 401 at its distal end, with respect to the carriage 400, of a foldable blade 402. The hooked end 401 is in abutting contact with the cradle 300 so as to limit its movement 4 of releasing the compressed drive spring 500 during translation thereof.

### Second Embodiment

In this embodiment, the two primary receptacles 600, 600′ are movable. The bottom of each primary receptacle 600, 600′ is static.

Referring now to FIG. 2B, a second embodiment of the drug preparation unit 200 according to the invention will be described. Thus, in FIG. 2B, an alternative to FIG. 2A is represented, and the elements common to the devices illustrated in FIGS. 2A and 2B that fill the same functions are assigned the same number. For clarity and conciseness, we will essentially describe hereinbelow the elements of the drug preparation unit 200 that differ from those previously described for the drug preparation unit 200 in FIG. 2A.

In FIG. 2B, the release mechanism 4 removably coupled to the compressed drive spring 500 comprises a stud 301 at its distal end, with respect to the carriage 400. The carriage 400 is an assembly indicated in FIG. 2B, it is able to receive a primary receptacle 600, 600′ therein.

In this embodiment, the stud 301 is retained by a hook 401′ able to rotate about an axis of rotation A2 to release it and enable the compressed drive spring 500 to move the second primary receptacle 600′, which is secured thereto, towards the needles 701, 701′. The first primary receptacle 600 is not represented. The stud 301 has a groove with a shape complementary to the opening of the hook 401′ enabling said hook 401′, when it is engaged, to block its movement of releasing the compressed drive spring 500. A release is obtained during the clockwise rotation of the hook 401′. The reconstituted mixture can be dispensed using an injection needle 721 after having circulated through the fluid circuit 700′.

The release of the compressed drive spring 500 is easier to understand with FIG. 2C. FIG. 2C shows that the compressed drive spring 500 is disposed between a first planar wall 302 belonging to the cradle 300 and a second planar wall 403 belonging to the carriage 400. Thus, during the release of the hook 401′ by clockwise rotation, the portion of the spring bearing on the first planar wall 302, in the upper position in FIG. 2C, remains under stress. At the other end of the drive spring 500, the latter relaxes, pushing on the second planar wall 403, also in the upper position in FIG. 2C, of the carriage 400 which contains the receptacle 600′, moving the latter towards the needles 701, 701′. The first planar wall 302 belonging to the cradle 300 and the second planar wall 403 belonging to the carriage 400 are parallel.

It is entirely possible to consider, according to the invention, an embodiment where the release is done by translation of the retaining element 401, 401′ (hooked end or stud) rather than a rotation.

Furthermore, in an embodiment where there would be only one receptacle able to be moved, this would imply that the second receptacle is pre-connected, i.e. fluidly connected beforehand with the fluid circuit. This embodiment is not discarded.

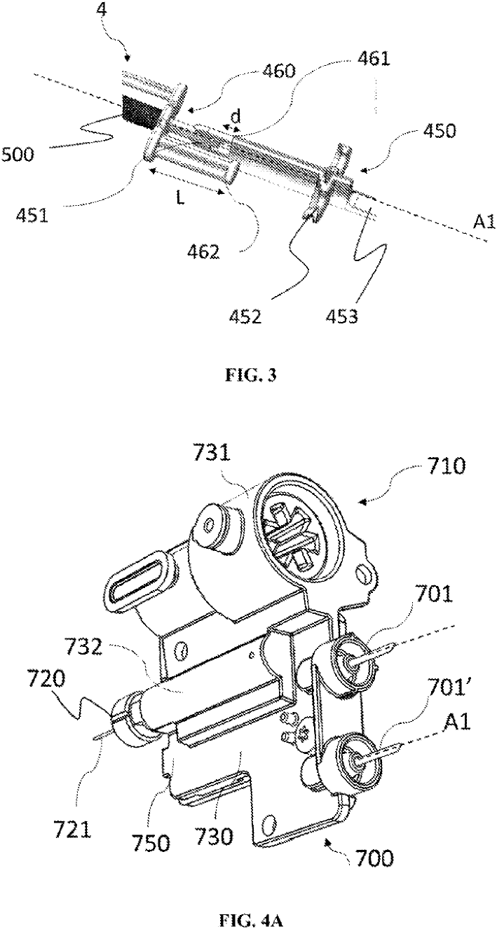

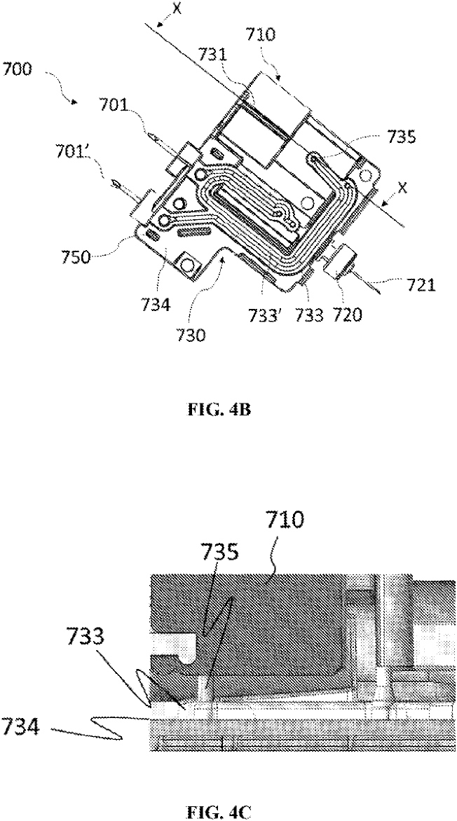

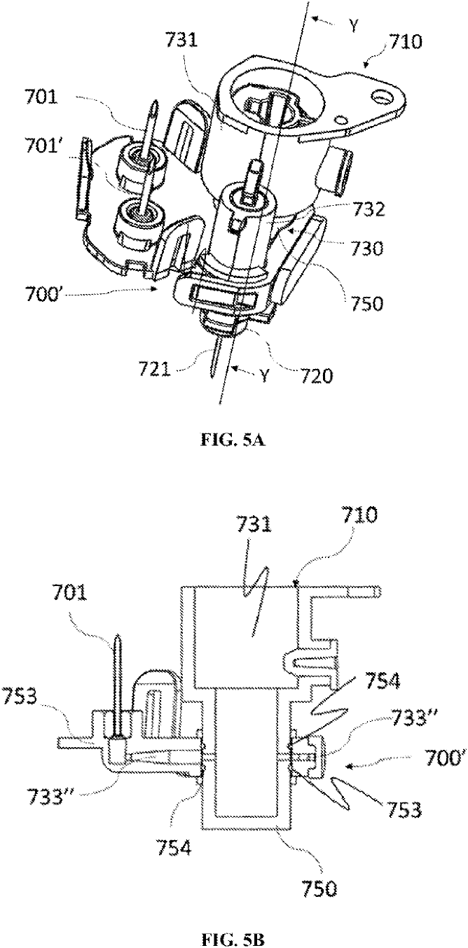

Reference is now made to FIG. 3 in which the release mechanism 4 according to the invention is partially represented. Indeed, FIG. 3 shows the mechanism leading to the movement of the primary receptacles 600, 600′ (not represented). The compressed drive spring 500 bears against an intermediate part 460. A receptacle support 450 extends along the axis A1 coinciding with that of extension of the compressed drive spring 500, the receptacle support 450 comprises two semi-circular openings 452 able to receive and retain primary receptacles 600, 600′ (not represented). The semi-circular openings 452 extend on either side of the extension axis A1 of the support and are intended to retain primary receptacles 600, 600′, not represented to facilitate understanding, by elastic stress. The movement of the primary receptacles 600, 600′ (not represented) towards the needles 701 is stopped by a stop 453 schematized in FIG. 3.

The intermediate part 460 is disposed between the receptacle support 450 and the compressed drive spring 500. The intermediate part 460 removably secures the receptacle support 450 to the compressed drive spring 500.

This fastening is done, in FIG. 3, using an elastic clamp 461 belonging to the receptacle support 450 and elastically retaining a first push rod 451 belonging to said intermediate part 460. The elastic clamp 461 is at the proximal end, with respect to the spring, of the receptacle support 450.

The axis of elongation of the push rod 451, the axis of the compressed drive spring 500, and the axis of elongation of the receptacle support 450 are coincident so that the force exerted by the spring is exerted at the elastic clamp. When the latter exceeds a given threshold F0, there is detachment between the intermediate part 460 and the receptacle support 450. It should be noted that the force needed to detach the intermediate part 460 and the receptacle support 450 is greater than or equal to the force needed to move at least one of the movable receptacles 600.

Thus, there is first a movement of the receptacle support 450, then contact with the support stop 453. Afterwards, there is detachment between the intermediate part 460 and the receptacle support 450 then, a distance d is able to be covered by the first push rod 451 which pushes against the receptacle support 450 to drive the latter and the receptacles 600 (not represented) that it would bring towards the needles 701 for fluid connection with the fluid circuit 700.

In a preferred embodiment of the release mechanism 4 according to the invention as illustrated in FIG. 3, the intermediate part 460 comprises a second push rod 462 extending laterally and parallel to the axis A1 so as to be able to exert a force on a possible shutter 620 (not represented) after detachment between the intermediate part 460 and the receptacle support 450.

The length L of the second push rod 462 is larger than the distance h separating the free end of the first push rod 451 and the stop surface on which said first push rod 451 bears to move the receptacle support 450. This also depends on the positioning height of the shutter 620 of the second receptacle 600′.

We will now describe a first embodiment of the fluid circuit 700 using FIGS. 4A, 4B and 4C. FIG. 4A which show in perspective view a coupling of a first planar fluid circuit 700 according to the invention with a pump 710 according to the invention.

FIG. 4A shows a fluid circuit 700 which is composed of a pump 710, a fluid selector 720 and a fluidic cassette 730. The planar fluidic cassette 730 is made of a central part 750 in which there is a pump barrel 731 for the pump 710, and a dispenser barrel 732 for the fluid selector 720.

In FIG. 4B, one could see that the fluid circuit 700 comprises a network of fluid channels 733, 733′ extending in a plane 734, the circuit being able to be fluidly connected with two primary receptacles 600, 600′ (not represented) and with the fluid selector 720 (not represented in FIG. 4B) via channels. This planar configuration allows reducing the bulk of the device 2 according to the invention. It should be noted that several embodiments of the fluid channels 733, 733′ can be used in the context of the invention and that the one given as example is in no way limiting.

The fluid channels 733, 733′ are visible at the main surface of the central part 750. Hence, the fluid channels 733, 733′ are mostly in a plane 734. These channels are closed by one or more sealing plate(s) assembled by welding or heading to the central part 750. The fluid channels 733, 733′, are connected to the pump barrel 731 and to the dispenser barrel 732 via connection channels 735 to the plane 734 containing the fluid channels 733, 733′. The fluidic cassette 730 may also be made by plastic injection.

FIG. 4C shows a section according to the axis XX of FIG. 4B, this figure allows better viewing the connection between the fluid channels 733 and the pump 710. The connecting channel 735 is shown therein extending perpendicular to the plane 734 of the planar fluidic cassette 730.

A second embodiment of the fluid circuit 700′ is illustrated using FIGS. 5A and 5B which show a perspective view and a plane cross-sectional view of a coupling of a second three-dimensional fluid circuit 700′ according to the invention with a pump 710 according to the invention.

In this second embodiment of the fluid circuit, the elements common to the devices illustrated in FIGS. 4A to 4C which perform the same functions are assigned the same number. For clarity and conciseness, we will essentially describe hereinbelow the elements of the fluid circuit that differ from those previously described for the first planar embodiment of the fluid circuit.

In FIG. 5A, the fluidic cassette 730 is made of a central part 750 that does not comprise fluid channels 733, 733′ like in the first planar embodiment. The fluid channels 733′ (cf. FIG. 5B) are located on peripheral parts 753 which are assembled to the central part by welding, clipping, or heading.

Sealing between the central part 750 and the peripheral parts 753 is achieved by compressed elastomeric seals 754. This embodiment allows having a three-dimensional fluid circuit which has the advantage of reducing bulk and the dead volumes in the fluidic path.

In general, and not related to a particular embodiment according to the invention, at least one needle 701, metallic or plastic, can be connected to the fluid channels 733, 733′, 733″ of the fluidic cassette 730. The needles 701, 701′ allow drawing the constituents contained in the primary receptacles 600, 600′.

The pump 710 may be a rotary pump, an oscillating-rotary pump, or a linear piston pump. The pump 710 may be mono-directional or bi-directional. The latter is actuated in order to move the fluid between two primary receptacles 600, 600′ and/or to move the fluid between a primary receptacle 600, 600′ and the delivery means 721 which may be a needle but also a sprayer for example. This has a significant advantage, as indicated hereinabove.

The fluid selector 720 is necessary for devices according to the invention allowing carrying out drug preparation and drug distribution or for devices according to the invention allowing carrying out reconstitution between primary receptacles 600, 600′ whose number is greater than or equal to two.

The fluid selector 720 is actuated in order to distribute the fluid between the different fluid channels 733, 733′, 733″. The fluid selector 720 may be rotary or linear. It includes at least two inlets and one outlet. The two inlets and the outlet are connected to a primary receptacle 600, 600′. An outlet may also be connected directly to an injection needle 721 or with a hose between the fluid selector 720 and the injection needle 721. In the case where the injection needle 721 is connected to the fluid selector 720 by a hose, it is possible to motorize the movement of the injection needle 721.

### Other Embodiments

The electromechanical assembly 900 (cf. FIGS. 2A and 2B) allowing connecting the fluid circuit is composed of at least one pump motor and one distributor motor when the actuation of the pump 710 and of the fluid selector 720 are dissociated. This solution with two motors allows for significant flexibility in developing a fluid recipe for mixing the drug. In this embodiment, the electromechanical assembly 900 comprises a pump motor and drive means. The pump motor allows activating only the pump 710 or, via a gear or a mechanical gearbox, controlling other elements (for example the distributor valves) for adjusting the circulation between the at least two primary receptacles 600, 600′ and/or the administration outlet.

In an alternative embodiment, the motors of the pump and of the fluid selector may be associated with a reduction gear. It is also possible to consider a pump docking system and a fluid selector docking system on the output axes of the pump and fluid selector motors. The pump and fluid selector docking systems may be plates, clips, suction cups, magnets, or bayonets. A single pump motor can be used if the movements of the pump and of the fluid selector are synchronized.

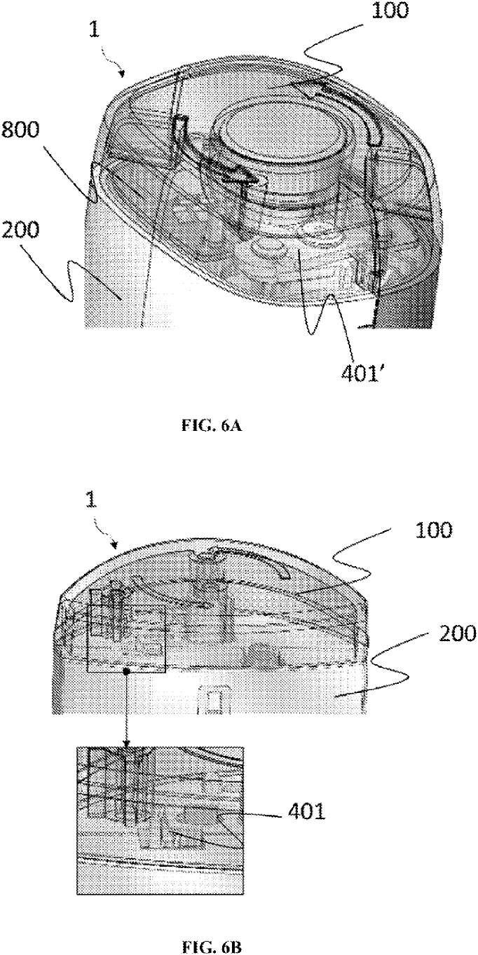

We will now describe in detail, thanks to FIGS. 6A and 6B, the activation unit 1 able to occupy an inactive position in which the compressed drive spring 500 is held and an active position in which the compressed drive spring 500 is released.

FIG. 6A shows an activation unit 1 comprising an electronic tray 800, a rotary button 100 and a hook 401′. The enlarged view in FIG. 6B shows the hooked end 401 projecting from the electronic tray 800.

### OPERATION

### First Embodiment

In this first embodiment, it is necessary to perform an action on the removable bottom of the second primary receptacle 600′ to facilitate the suction by the motor pump 710 of the second primary receptacle 600′ regardless of the position of the device 2. The device 2 according to the first embodiment has the advantage of not requiring complete push on the second primary receptacle 600′ but only “breaking” the sticking forces between the walls of the second primary receptacle 600′ and its removable bottom. These sticking forces may be greater with ageing of the device 2.

### Second Embodiment

Thanks to the electromechanical assembly 900, the device 2 according to the present invention enables a decorrelation between the drug reconstitution and the injection. There is sequencing of the operations, which allows eliminating any risk of administration without being assured of the optimal reconstitution of the drug.

The operating kinematics of the second embodiment (that of FIG. 2B) will now be detailed with reference to the different parts detailed hereinabove:

When the activation button 100 is activated by a user by a rotation or a translation, it moves the foldable blade 402, thereby releasing the retaining element 401, 401′.

As illustrated in FIG. 2B, it is also possible to release the retaining element 401, 401′ via the hooked end 401.

This action will release the compressed drive spring 500 thus generating a translational movement of the carriage 400 with the primary receptacles 600, 600′ in the cradle 300 towards the needles 701, 701′ (cf. FIGS. 2A and 2B). At the end of its stroke, each primary receptacle 600, 600′ is connected to the fluid circuit 700 through a needle 701, 701′ passing through the corresponding septum 601, 601′ of the receptacle 600, 600′. According to the invention, the spring 500 will be sized so as to have a sufficiently high stiffness to pierce the septum 601, 601′ by the needle 701, 701′.

In a preferred embodiment, the drug delivery process is activated once the receptacle(s) is or are connected to the fluid circuit 700.

In order to trigger this delivery process, during the movement of the carriage 400 or at the end of its movement, the carriage 400 can wake up the electronics of the device according to the invention by actuating an electronic switch. The electronic switch may be a contact switch or an optical switch or possibly a magnetic switch.

Of course, the device comprises a battery, when the electronic switch is actuated, the battery can be connected to the electronics. Depending on the selected electronic switch, the battery may be: either partially connected to the electronics, which means that a slight electric current will be consumed by the electronics, or completely disconnected from the electronics, which means that there will be no power consumption, thereby lengthening the service life of the battery. In the case of a reusable device, it will be preferable to use a rechargeable battery.

It is essential to note that the two embodiments disclosed regarding the release mechanism 4 and the two embodiments regarding the planar and three-dimensional fluid circuits are mechanically independent and can therefore be combined to obtain configurations of the device according to the invention.

The device according to the invention intends the combination of the preparation and the administration by injection of drug products. If the subcutaneous pathways of a patient have been primarily explored, it is possible to consider to the least the intravenous and intramuscular pathways as other pathways.

The device according to the invention enables mixing between a constituent A and a constituent B. If the constituent A is the solvent and the constituent B the pharmaceutical active substance, it is possible to consider, as already mentioned, filling the first receptacle 600 with a body fluid: blood, lymph, or other non-circulating biological liquids (synovial, cerebrospinal, pleural...), for extra-corporeal homogenization of the active substance in a target liquid, for re-administrations via the appropriate injection mode according to the invention.

It is also possible to use the device according to the invention for mixing two liquids: in this case we will talk about a co-injection device. The field of application preferably remains healthcare and the medical sector.

### REFERENCE NUMERALS

1: Activation unit
2: Device according to the invention
200: Drug preparation
300: Cradle
301: Hook
302: First planar wall of the cradle
4: Release mechanism
400: Carriage
401, 401′: Retaining element
402: Foldable Blade
403: Second planar wall of the carriage
450: Receptacle support
451: First push rod
452: Receptacle fastening elastic means
453: Support stop
460: Intermediate part
461: Elastic clamp
462: Second push rod
500: Drive spring
600, 600′: Receptacle
601, 601′: Septum
620: Shutter
700, 700′: Fluid circuit (planar or three-dimensional)
701, 701′: Piercing means
710: Motor pump
720: Fluid selector
721: Administration means
730: Fluidic cassette
731: Pump barrel
732: Barrel of the fluid selector
733, 733′, 733″: Fluid channels
734: Plane of the fluidic cassette
735: Link channel
750: Central part of the fluid circuit
753: Peripheral parts
754: Compressed elastomeric seals
800: Electronic tray
A1: Main axis of the device (axis of the receptacles, of the spring, of the needles)
C: Shell
d: Distance between the first push rod and the receptacle support before fluid connection with the fluid circuit.
L: Length of the second push rod

1. Device (2) for drug preparation and drug delivery comprising:
-a drug preparation unit (200) comprising at least two containers (600, 600′) capable of containing constituents to be mixed, at least one of the containers (600, 600′) being mobile in said drug preparation unit (200),
-a means of administration (721) of the drug, such as an injection or spray means,
-a fluidic circuit (700,700′) able to put in fluidic connection the at least two containers (600, 600′) and the administration means (721), the connection of the fluidic circuit (700, 700′) with the at least two containers (600, 600′) being made through at least two piercing means (701) when said at least two containers (600, 600′) move from an initial position to a contact position with said at least two corresponding drilling means (701),
-an electromechanical assembly (900) for the fluidic drive of the constituents within the fluidic circuit (700, 700′), the electromechanical assembly (900) being able to activate the fluidic circuit (700, 700′), by means of′ a motorized pump (710), characterized in that said drug preparation unit (200) comprises a release mechanism (4) comprising a compressed driving spring (500) which, upon its release, is able to generate a displacement along an axis (A1) of at least one of the mobile containers (600, 600′) towards at least one of said drilling means (701) to create the fluidic connection with the fluidic circuit (700, 700′).

2. Device (2) for drug preparation and drug delivery according to claim 1 further comprising:
-a motorized pump (710) connected to said fluidic circuit (700, 700′) to circulate at least one of the constituents in said fluidic circuit (700, 700′) once the fluidic connection has been created.

3. Device (2) for drug preparation and drug delivery according to claim 1 wherein the drug preparation unit (200) comprises:
-a cradle (300) comprising a first flat wall (302) perpendicular to an axis A1 parallel to the movement of said containers (600, 600′) and,
-a cart (400) movably mounted in said drug preparation unit and abutting against at least one of the containers (600, 600′), said cart (400) comprising a second flat wall (403) parallel to the first flat wall (302) of the cradle (300),
-said drive spring (500) being compressed between said first flat wall (302) of the cradle (300) and said second flat wall (403) of the carriage (400) so that the release of the compressed drive spring moves the at least one container (600, 600′) through the carriage (400) to at least one of said piercing means (701).

4. Device (2) for drug preparation and drug delivery according to claim 1 wherein the release mechanism (4) comprises:
-a container support (450) extending in the direction Al coaxially with the compressed drive spring (500), said support (450) comprising at least two elastic fixing means (452) of said containers (600),
-an intermediate piece (460) removably securing the container support (450) to the compressed drive spring (500),
-said joining being done using an elastic clamp (461) belonging to the container support (450) and resiliently retaining a first push rod (451) belonging to said intermediate part (460), so that the force exerted by the spring required to separate the intermediate piece (460) and the container support (450) is greater than or equal to the force required to move at least one of the mobile containers (600, 600′).

5. Device (2) for drug preparation and drug distribution according to claim 4 wherein:
-at least one of the two containers (600, 600′) is closed by a shutter (620) capable of moving under the effect. an external thrust in order to expel its constituent, and in which,
-said intermediate piece (460) comprises a second push rod (462) extending parallel to the axis (Al) of pushing the containers along said axis (Al) of the shutter (620) in order to exert a force on said shutter (620) after separation between the intermediate part (460) and the container support (450).

6. Device (2) for preparing medicament and dispensing medicament according to claim 1 comprising an activation unit (1) capable of occupying an inactive position in which the compressed driving spring (500). is maintained and an active position in which the compressed drive spring (500) is released.

7. Device (2) for drug preparation and drug delivery according to claim 6 wherein the activation unit (1) comprises a retaining element (401,401′) capable of being driven by said activation unit (1) from a position of holding the spring (500) in its compressed state to a position of release of the spring (500), said release position of the spring (500) being obtainable by a rotation or a translation of said unit of activation (1) by a user.

8. Device (2) for drug preparation and drug distribution according to claim 6 wherein the activation unit (1) comprises an electronic card provided with an electronic switch of optical, magnetic, or contact type, able to wake up the electromechanical assembly for the motorized drive of the pump (710).

9. Device (2) for drug preparation and drug delivery according to claim 1 wherein the fluidic circuit (700) comprises a network of fluidic channels (733,733′) extending in a plane (734) and in connection with the at least two containers (600, 600′) and the administration means (721) via channels to reduce the bulk in the device (2).

10. Device (2) for drug preparation and drug delivery according to claim 1 wherein the fluidic circuit (700′) comprises a network of fluidic channels (733”) extending at the periphery of a central part (750) to reduce bulk and reduce dead volumes in the fluid path.

11. Device (2) for drug preparation and drug distribution according to claim 1 such as a first assembly comprising the fluid circuit (700,700′), the at least two containers (600, 6000′) and the administration means is disposable while a second assembly comprising the release mechanism (4), motorized pump (710) and the electromechanical assembly (900) for its training is reusable.

12. Device (2) for drug preparation and drug delivery according to claim 1 comprising a fluid selector (720) capable of selectively connecting the at least two containers (600, 600′) and the means of administration (721).

13. A method of automated preparation and distribution of a medicament using a device according to claim 1 and comprising the following steps:
-providing a medicament preparation unit (200) comprising at least two containers (600, 600′), one comprising a drug in hydrated or dehydrated form and the other a solvent,
-providing a means of administration (721) of the medicament, such as an injection or spray means, -provide a fluidic circuit (700,700′) able to put in fluidic connection the at least two containers (600, 600′) and the administration means (721), the connection of the fluidic circuit (700, 700′) with the at least two containers (600) being made through at least two piercing means (701) when said at least two containers (600, 600′) move from an initial position to a position of contact with said at least two corresponding drilling means (701),
-providing an electromechanical assembly (900) for the fluidic entrainment of the constituents within the fluidic circuit (700, 700′), characterized in that the automated preparation and dispensing of the drug comprises the release of a release mechanism (4 ) comprising a compressed drive spring (500) which, when released, generates a displacement along an axis (Al) of at least one of the mobile containers (600, 600′) towards at least one of said piercing means (701) to create the fluidic connection with the fluidic circuit (700, 700′).

14. A method of automated preparation and dispensing of a medicament according to claim 13, in which electronic activation causes the fluidic entrainment of the constituents within the fluidic circuit and the administration of the medicament.

15. The method of automated preparation and dispensing of a medicament according to claim 14, in which the electronic activation takes place during the movement of at least one of the mobile containers (600, 600′) by means of a switch on. the path of movement of said containers.
